(19) United States
(12) Patent Application Publication
(10) Pub. No.: US 2007/0149916 A1
(43) Pub. Date: Jun. 28, 2007

(54) Dry matrices as drug reservoirs in electrotransport applications

(75) Inventors: Janardhanan A. Subramony, Santa Clara, CA (US); Rama V. Padmanabhan, Los Altos, CA (US); Joseph B. Phipps, Sunnyvale, CA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: ALZA CORPORATION, Mountain View, CA (US)

(21) Appl. No.: 11/613,327

(22) Filed: Dec. 20, 2006

Related U.S. Application Data
(60) Provisional application No. 60/753,359, filed on Dec. 22, 2005.

(51) Int. Cl. A61N 1/30; A61K 38/16; A61K 31/573; A61K 31/473; A61K 31/445; A61K 31/24

(52) U.S. Cl. 604/20; 514/12; 514/171; 514/534; 514/317; 514/289

(57) ABSTRACT

The present invention provides methods and devices for the electrotransport delivery of beneficial agents that utilize polymer electrolyte matrices as drug reservoirs. In certain aspects of the invention, the beneficial agents are hydrolytically unstable, and methods are provided for enhancing the stability of the hydrolytically unstable beneficial agents during long-term storage of devices for the electrotransport delivery of the hydrolytically unstable beneficial agents and during electrotransport delivery of the hydrolytically unstable beneficial agents.

### CROSS REFERENCE TO RELATED APPLICATIONS

This application claims the benefit of U.S. application Ser. No. 60/753,359, filed Dec. 22, 2005, which is incorporated herein by reference in its entirety.

### FIELD OF THE INVENTION

The present invention relates to devices and methods for the electrotransport delivery of beneficial agents that utilize polymer electrolyte reservoirs. In certain aspects of the invention, the beneficial agents are hydrolytically unstable, but remain stable during storage of the electrotransport devices and during electrotransport delivery.

### BACKGROUND OF THE INVENTION

The transdermal delivery of therapeutic agents by diffusion through the epidermis offers certain improvements over more traditional drug delivery methods, such as subcutaneous injection and oral delivery. Transdermal drug delivery avoids the hepatic first pass effect encountered with oral drug delivery, and also eliminates patient discomfort associated with subcutaneous injections. In addition, transdermal delivery can provide more uniform concentrations of a drug in the bloodstream of the patient over time due to the extended controlled delivery profiles of certain types of transdermal delivery devices.

The skin functions as the primary barrier to the transdermal penetration of materials into the body and represents the body's major resistance to the transdermal delivery of therapeutic agents such as drugs. To date, efforts have focused on reducing the physical resistance or enhancing the permeability of the skin for the delivery of drugs by passive diffusion. Various methods for increasing the rate of transdermal drug flux have been attempted, most notably using chemical flux enhancers. Other approaches for increasing the rate of transdermal drug delivery include the use of energy sources, such as electrical energy and ultrasonic energy, to electrically assist the transdermal delivery of therapeutic agents.

Hydrophillic polymer-based gels, or hydrogels, are commonly used as drug reservoirs in electrotransport drug delivery devices. Hydrogels typically contain approximately 80% water in their final, processed form that contains the therapeutic agent, and the water provides a conduction medium and pathway for the transport of the agent via electrotransport. Hydrogels are therefore excellent biocompatible reservoirs for therapeutic agents that have sufficient aqueous stability. Chemical stability problems can arise, however, when hydrolytically unstable therapeutic agents are formulated in hydrogels for electrotransport delivery. Such stability problems can arise both during electrotransport delivery and during long-term storage of the delivery devices. Furthermore, in electrotransport devices in which the electronics and the therapeutic agent formulation are assembled in a single compartment, the electronics can be negatively affected by the moisture and relative humidity associated with hydrogels. There is thus a need in the art for drug reservoirs for electrotransport drug delivery devices that can be used with hydrolytically unstable therapeutic agents and that do not negatively affect the electronic components of the devices.

### SUMMARY OF THE INVENTION

Certain aspects of the present invention relate to devices for the electrotransport delivery of beneficial agents that comprise a donor electrode assembly comprising a donor reservoir that comprises a substantially solvent-free polymer electrolyte, a counter electrode assembly, and a source of electrical power connected to the donor and counter electrode assemblies. In preferred embodiments of the invention, the polymer electrolyte is substantially free of oxidants and ionic impurities and contains a beneficial agent that remains stable during long-term storage of the device and during electrotransport.

Other aspects of the present invention relate to methods for enhancing the stability of hydrolytically unstable beneficial agents during long-term storage of devices for the electrotransport delivery of hydrolytically unstable beneficial agents and during electrotransport delivery of hydrolytically unstable beneficial agents. Such methods preferably comprise providing a device for the electrotransport delivery of hydrolytically unstable beneficial agents that comprises a donor electrode assembly comprising a donor reservoir that comprises a polymer electrolyte matrix that is substantially free of oxidants and ionic impurities and contains the hydrolytically unstable beneficial agent; a counter electrode assembly; and a source of electrical power adapted to be electrically connected to the donor and counter electrode assemblies. In preferred aspects, such methods further comprise storing the devices for up to six months; and administering the hydrolytically unstable beneficial agent to a patient using the device, wherein the hydrolytically unstable beneficial agent remains stable during storage and during electrotransport.

### BRIEF DESCRIPTION OF THE DRAWINGS

FIG. 1 depicts the in vitro flux of lidocaine HCl in various matrices.

FIG. 2 is an HPLC chromatogram that demonstrates improved stability of hydrolytically labile hydrocortisone hemisuccinate (HCHS) in polyethylene oxide (PEO) matrices as compared to polyvinyl alcohol (PVOH) hydrogels.

FIG. 3A shows the results of stability studies of HCHS in PVOH hydrogels.

FIG. 3B shows the results of stability studies of HCHS in PEO films.

FIG. 4 shows the stability of apomorphine in various PEO matrices. PEO20-200K: the molecular weight (MW) of PEO used was 200K and the ratio of PEO to drug was 20; PEO10-7000K: the MW of PEO used was 7000K and the ratio of PEO to drug was 10; PEO20-7000K: the MW of PEO used was 7000K and the ratio of PEO to drug was 20.

FIG. 5A shows a comparison of apomorphine in vitro transdermal electrotransport flux in PVOH and PEO matrices as a function of time showing the rise to steady state and the steady state profile.

FIG. 5B shows the steady state average flux values for the in vitro transdermal electrotransport flux of apomorphine in PVOH and PEO matrices.

FIG. 6 shows the in vitro flux of fentanyl HCl (μg/cm²hr) in a PEO matrix.

FIG. 7 shows a comparison of the in vitro flux of fentanyl HCl in a PEO matrix with that of fentanyl HCl in a PVOH hydrogel. PVOH-Fentanyl R is a repeat study.

FIG. 8 is a perspective exploded view of an electrotransport drug delivery device in accordance with certain aspects of the present invention.

### DETAILED DESCRIPTION OF ILLUSTRATIVE EMBODIMENTS

Certain aspects of the present invention relate to polymer electrolyte matrices that can be used as drug reservoirs in electrotransport drug delivery devices. Polymer electrolytes are solvent-free, ion-conducting polar polymers that can transport charged molecules and ions. Polymer electrolytes contain cation coordinating sites, such as polar groups having lone pair electrons, have a highly amorphous morphology, and have low glass transition temperatures leading to highly flexible polymer backbones.

The ionic conductivity and ion transport properties of polymer electrolytes are due to the large amplitude segmental motion of the polymers that occurs upon electrical perturbation. Polymer electrolytes include polyethylene oxide, whose ability to act as an electrolyte to transport cations has been studied in detail in connection with electrochemical devices such as batteries, gas sensors, and fuel cells. Substantially solvent-free polymer electrolyte matrices make ideal reservoirs for electrotransport devices used for the delivery of therapeutic agents that are hydrolytically unstable during long-term storage and during electrotransport. The use of dry polymer electrolyte reservoirs for electrotransport devices also eliminates problems, such as corrosion or electrical shorting, that can arise when humidity from hydrated reservoirs penetrates the electronic components of electrotransport devices. Furthermore, the use of dry polymer electrolyte reservoirs with low ohmic resistance eliminates the extra step of hydration that is required when hydratable donor matrices are used. In addition, polymer electrolyte matrices facilitate miniaturization of electrotransport beneficial agent delivery devices, particularly the beneficial agent reservoir.

Certain aspects of the present invention relate to dry, substantially solvent-free polymer electrolyte matrices for electrotransport drug delivery devices that are in the form of thin films.

As used herein, the terms “electrotransport,” “iontophoresis,” and “iontophoretic” refer to the delivery of pharmaceutically active agents (charged, uncharged, or mixtures thereof) through a body surface (such as skin, mucous membrane, eye, or nail) wherein the delivery is at least partially induced or aided by the application of an electric potential. The agent may be delivered by electromigration, electroporation, electroosmosis or any combination thereof. Electromigration (also called iontophoresis) involves the electrically induced transport of charged ions through a body surface. Electroosmosis has also been referred to as electrohydrokinesis, electro-convection, and electrically induced osmosis. In general, electroosmosis of a species into a tissue results from the migration of solvent in which the species is contained, as a result of the application of electromotive force to the therapeutic species reservoir, i.e., solvent flow induced by electromigration of other ionic species. During the electrotransport process, certain modifications or alterations of the skin may occur such as the formation of transiently existing pores in the skin, also referred to as “electroporation.” Any electrically assisted transport of species enhanced by modifications or alterations to the body surface (e.g., formation of pores in the skin) are also included in the term “electrotransport” as used herein. Thus, as used herein, the terms “electrotransport,” “iontophoresis” and “iontophoretic” refer to (1) the delivery of charged drugs or agents by electromigration, (2) the delivery of uncharged drugs or agents by the process of electroosmosis, (3) the delivery of charged or uncharged drugs by electroporation, (4) the delivery of charged drugs or agents by the combined processes of electromigration and electroosmosis, and/or (5) the delivery of a mixture of charged and uncharged drugs or agents by the combined processes of electromigration and electroosmosis.

In electrotransport devices, at least two electrodes are used. Both of the electrodes are disposed so as to be in intimate electrical contact with some portion of the skin, nails, mucous membrane, or other surface of the body. One electrode, called the “active” or “donor” electrode, is the electrode from which the drug is delivered into the body. The other electrode, called the “counter” or “return” electrode, serves to close the electrical circuit through the body. In conjunction with the patient's skin, the circuit is completed by connection of the electrodes to a source of electrical power, e.g., a battery, and usually to circuitry capable of controlling current passing through the device. If the ionic substance to be driven into the body is positively charged, then the positive electrode (the anode) will be the donor electrode and the negative electrode (the cathode) will serve as the counter electrode, completing the circuit. If the ionic substance to be delivered is negatively charged, then the cathodic electrode will be the donor electrode and the anodic electrode will be the counter electrode. Both the anode and the cathode can be donor electrodes if both anionic and cationic therapeutic agent ions are to be delivered, or if an uncharged therapeutic agent is to be delivered.

Electrotransport devices additionally require a reservoir or source of the pharmaceutically active agent that is to be delivered or introduced into the body, referred to as the “donor reservoir.” Examples of donor reservoirs include a pouch or cavity, a porous sponge or pad, and a hydrophilic polymer or gel matrix. Such drug reservoirs are connected to, and positioned between, the donor electrode of the electrotransport device and the body surface, to provide a fixed or renewable source of one or more desired species or agents. The term “donor electrode assembly” thus refers to the donor electrode and the donor reservoir. Similarly, the term “counter electrode assembly” refers to the counter electrode and the counter reservoir, which contains one or more biocompatible electrolytes.

Electrotransport devices are powered by an electrical power source such as one or more batteries. Typically, at any one time, one pole of the power source is electrically connected to the donor electrode, while the opposite pole is electrically connected to the counter electrode. Since it has been shown that the rate of electrotransport drug delivery is approximately proportional to the electric current applied by the device, many electrotransport devices typically have an electrical controller that controls the voltage and/or current applied through the electrodes, thereby regulating the rate of drug delivery. These control circuits use a variety of electrical components to control the electrical signal, i.e., the amplitude, polarity, timing, waveform shape, etc. of the electric current and/or voltage, supplied by the power source. U.S. Pat. No. 5,047,007 to McNichols, et al., which is hereby incorporated by reference in its entirety, discloses several suitable parameters and characteristics.

An electrotransport device or system, with its donor and counter electrodes, may be thought of as an electrochemical cell having two electrodes, each electrode having an associated half cell reaction, between which electrical current flows. Electrical current flowing through the conductive (e.g., metal) portions of the circuit is carried by electrons (electronic conduction), while current flowing through the liquid-containing portions of the device (i.e., the drug reservoir in the donor electrode, the electrolyte reservoir in the counter electrode, and the patient's body) is carried by ions (ionic conduction). Current is transferred from the metal portions to the liquid phase by means of oxidation and reduction charge transfer reactions that typically occur at the interface between the metal portion (e.g., a metal electrode) and the liquid phase (e.g., the drug solution). A detailed description of the electrochemical oxidation and reduction charge transfer reactions of the type involved in electrically assisted drug transport can be found in electrochemistry texts such as J. S. Newman, Electrochemical Systems (Prentice Hall, 1973) and A. J. Bard and L. R. Faulkner, Electrochemical Methods, Fundamentals and Applications (John Wiley & Sons, 1980).

As used herein, the terms “transdermal administration” and “transdermally administering” refer to the delivery of a substance or agent by passage into and through the skin, nails, mucous membrane, or other surface of the body.

As used herein, the term “matrix” refers to a porous, composite, solid, or semi-solid substance, such as, for example, a polymeric material or a gel, that has pores or spaces sufficiently large for a beneficial agent to populate. The matrix serves as a repository in which the beneficial agent is contained.

As used herein, the phrase “substantially free of oxidants and impurities” refers to polymer electrolytes that contain no more than a trace or trivial amount of oxidants and ionic impurities.

As used herein, the phrase “long-term storage” refers to the storage of an electrotransport beneficial agent delivery device for a period of time that is at least two weeks. For example, storage of electrotransport delivery devices for periods of time that would be considered “long-term” include storage for at least one month, at least three months, at least six months, or at least twelve months.

As used herein, the term “beneficial agent” refers to any agent that elicits a desired beneficial, often pharmacological, effect upon administration to a human or animal, whether alone or in combination with other pharmaceutical excipients or inert ingredients.

As used herein, the term “polymer electrolyte” refers to any polymeric material that is capable of conducting ions. Polymer electrolytes can be substantially free of solvents, or can contain trace amounts of the solvents that are used to cast films of the polymer electrolytes.

As used herein, the term “hydrolytically unstable” refers to substances that undergo degradation by hydrolysis. The term “hydrolysis” refers to any chemical process by which a molecule is cleaved into two or more parts by the addition of water.

As used herein, the phrase “beneficial agent that remains stable” refers to a beneficial agent that remains substantially intact and does not undergo hydrolysis to a significant degree during long-term storage of an electrotransport delivery device containing the beneficial agent and during electrotransport delivery of the beneficial agent. The term “stability,” as used herein, refers to the extent that a beneficial agent is resistant to hydrolysis.

As used herein, the phrase “method for enhancing the stability of a hydrolytically unstable beneficial agent” refers to methods that result in any measurable increase in the stability of a hydrolytically unstable beneficial agent.

As used herein, the term “thin film” refers to polymer electrolyte films that are from approximately 0.2 mm to approximately 2.0 mm thick. In certain embodiments of the invention, layers of thin polymer electrolyte films can be used to obtain polymer electrolyte films that are approximately 1.59 mm thick.

Particular aspects of the present invention relate to devices for the electrotransport delivery of beneficial agents. In preferred embodiments of the invention, the devices comprise a donor electrode assembly that comprises a donor reservoir comprising a polymer electrolyte that is substantially free of oxidants and impurities and contains a beneficial agent that remains stable during long-term storage of the device and during electrotransport. Preferably, the devices further comprise a counter electrode assembly and a source of electrical power connected to the donor and counter electrode assemblies.

Other aspects of the invention relate to methods for enhancing the stability of hydrolytically unstable beneficial agents during long-term storage of devices for the electrotransport delivery of hydrolytically unstable beneficial agents and during electrotransport delivery of hydrolytically unstable beneficial agents. In preferred embodiments of the invention, such methods comprise providing a device for the electrotransport delivery of hydrolytically unstable beneficial agents that comprises a donor electrode assembly. The donor electrode assembly preferably comprises a donor reservoir comprising a polymer electrolyte that is substantially free of oxidants and impurities and contains the hydrolytically unstable beneficial agent. The device further preferably comprises a counter electrode assembly and a source of electrical power connected to the donor and counter electrode assemblies. The methods further preferably comprise storing the device for up to six months and administering the hydrolytically unstable beneficial agent to a patient using the device. In preferred aspects of the invention, the hydrolytically unstable beneficial agent remains stable during storage and during electrotransport.

In certain aspects of the methods of the invention, a device for the electrotransport delivery of a hydrolytically unstable beneficial agent is stored for up to six months prior to the electrotransport delivery of the hydrolytically unstable beneficial agent, and the beneficial agent remains stable throughout the time in which it is stored in the electrotransport delivery device. In other aspects of the invention, a device for the electrotransport delivery of a hydrolytically unstable beneficial agent is stored for any period of time of at least six months, and the beneficial agent remains stable throughout the period of time in which it is stored in the electrotransport delivery device.

In preferred embodiments of the devices and methods of the present invention, the beneficial agent delivered via electrotransport has a net positive or negative charge. In other embodiments of the invention, the beneficial agent is hydrolytically unstable and undergoes hydrolysis or structural degradation upon exposure to water. In certain aspects of the invention, the hydrolytically unstable beneficial agent is a hydrolytically unstable protein or polypeptide.

Particular embodiments of the invention relate to electrotransport devices and methods in which the beneficial agent delivered via electrotransport is lidocaine hydrochloride, hydrocortisone hemisuccinate, apomorphine hydrochloride, or fentanyl hydrochloride. In other embodiments of the invention, the beneficial agent is leutinizing hormone releasing hormone (LHRH), an LHRH analog (such as goserelin, leuprolide, buserelin, triptorelin, gonadorelin, and napfarelin, a menotropin (urofollitropin (FSH) and LH)), vasopressin, desmopressin, corticotrophin (ACTH), an ACTH analog such as ACTH (1-24), calcitonin, vasopressin, deamino[Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, an interferon, an interleukin, a menotropin (urofollitropin (FSH) and LH), oxytocin, streptokinase, tissue plasminogen activator, urokinase, ANP, ANP a clearance inhibitor, BNP, VEGF, an angiotensin II antagonist, an antidiuretic hormone agonist, a bradykinin antagonist, ceredase, a CSI, calcitonin gene related peptide (CGRP), an enkephalin, a FAB fragment, IgE a peptide suppressor, IGF-1, a neurotrophic factor, a colony stimulating factor, a parathyroid hormone and agonist, a parathyroid hormone antagonist, a prostaglandin antagonist, pentigetide, protein C, protein S, a renin inhibitor, thymosin alpha-1, a thrombolytic, TNF, a vasopressin antagonist analog, alpha-I antitrypsin (recombinant), TGF-beta, fondaparinux, ardeparin, dalteparin, defibrotide, enoxaparin, hirudin, nadroparin, reviparin, tinzaparin, pentosan polysulfate, an oligonucleotides and oligonucleotide derivative such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanil, sufentanil, alfentanil, lofentanil, carfentanil, and mixtures thereof.

In particular aspects of the present invention, the electrotransport beneficial agent delivery devices comprise a donor electrode assembly that comprises a donor reservoir comprising a polymer electrolyte. The polymer electrolyte is substantially free of oxidants and impurities. In particular embodiments, polymer electrolytes that contain antioxidants are preferred.

In certain embodiments of the invention, the polymer electrolyte that comprises the donor reservoir of an electrotransport beneficial agent delivery device is in the form of a thin film, and the polymer electrolyte is preferably polyethylene oxide, a polysiloxane having a hydrophilic side chain, or a polyphosphazene having a hydrophilic side chain. In particularly preferred embodiments of the invention, the polymer electrolyte is polyethylene oxide. Polyethylene oxide is available in a variety of molecular weights (100,000 to 8×10⁶), and polyethylene oxide with a molecular weight of 4×10⁶ having 200 to 500 ppm of the antioxidant butylated hydroxytoluene (BHT) is particularly preferred. Polysiloxanes have flexible Si—O backbones, as seen in the structure below. Upon substituting a hydrophilic side chain, such as, for example, an ethoxy or a methoxy group, for R, polysiloxanes can function as polymer electrolytes. A preferred siloxane-based polymer electrolyte is polydimethyl siloxane (PDMS) in which R is substituted with a hydroxyl, methoxy, or ethoxy group. 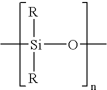
Polyphosphazenes (shown in the structure below) that contain hydrophilic side chains, such as methoxy or ethoxy, function as polymer electrolytes and exhibit improved conductivity due to the flexible phosphazene backbone.

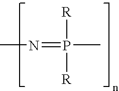

In preferred aspects of the invention, polymer electrolytes used as donor reservoirs in electrotransport beneficial agent delivery devices are prepared by a method known as solution casting in which a solution containing the dry form of a polymer electrolyte is first dissolved in a solvent. Solvents that can be used for solution casting include organic solvents that have high vapor pressures or low normal boiling points and have received regulatory approval as pharmaceutical solvents suitable for transdermal administration. Non-aqueous solvents are preferred in cases where the beneficial agent is hydrolytically unstable. Preferred solvents include, for example, water, acetonitrile, methanol, ethanol, lower alkyl alcohols such as isopropyl alcohol, acetone, methyl ethyl acetone, and heptane, either alone or in combination.

Once the polymer electrolyte is dissolved in a solvent, the required amount of beneficial agent, based upon the desired molar ratio of the beneficial agent to the polymer electrolyte, is then added. The ratio of the beneficial agent to the polymer electrolyte is typically expressed in terms of the number of drug molecules per polar group (such as oxygen) in the backbone of the polymer electrolyte. Typical polymer electrolyte beneficial agent ratios range from 5 to 25, depending upon the beneficial agent and the beneficial agent loading required. Higher beneficial agent concentrations can induce crystallinity in the resulting film, which has an unfavorable impact on conductivity.

The solution containing the beneficial agent and the polymer electrolyte is then heated to a temperature in the range of 40° C. to 60° C. (a temperature below the boiling point of the solvent used for casting), and cast into molds. The molds are typically made of delrin or Teflon, and their dimensions can be designed to yield films of a desired thickness. The solvent used for casting is then removed either by application of a vacuum or by mild heating, resulting in thin flexible films of a beneficial-agent containing polymer electrolyte.

Beneficial agent-containing polymer electrolyte films can alternatively be prepared by first forming a polymer electrolyte film according to the procedures described above, except that the beneficial agent is omitted. A beneficial agent dissolved in a suitable solvent can then be imbibed into the resultant film.

Polymer electrolytes can be used as donor reservoirs in any suitable electrotransport beneficial agent delivery device. A suitable electrotransport device includes an anodic donor electrode, preferably comprised of silver, and a cathodic counter electrode, preferably comprised of silver chloride. The donor electrode is in electrical contact with the polymer electrolyte donor reservoir that contains the beneficial agent. The counter reservoir can comprise any conductive electrolyte, such as, for example, a polyvinyl alcohol gel, and contains a biocompatible electrolyte, such as citrate buffered saline. The anodic and cathodic reservoirs preferably each have a skin contact area of about 1 to 5 cm² and more preferably about 2 to 3 cm². The anodic and cathodic reservoirs preferably have a thickness of about 0.05 to 0.25 cm, and more preferably about 0.15 cm. The applied electrotransport current is about 150 μA to about 240 μA. Most preferably, the applied electrotransport current is substantially constant direct current during the dosing interval.

The cathodic electrode and the anodic electrode are comprised of electrically conductive material such as a metal. For example, the electrodes can be formed from a metal foil, a metal screen, or metal deposited or painted on a suitable backing, or by calendaring, film evaporating, or mixing the electrically conductive material in a polymer binder matrix. Examples of suitable electrically conductive materials include carbon, graphite, silver, zinc, aluminum, platinum, stainless steel, gold and titanium. For example, as noted above, the anodic electrode can be composed of silver which is also electrochemically oxidizable. The cathodic electrode can be composed of carbon and electrochemically reducible silver chloride. Silver is preferred over other metals because of its relatively low toxicity to mammals. Silver chloride is preferred because the electrochemical reduction reaction occurring at the cathode (AgCl+e⁻→Ag^{o}+Cl⁻) produces chloride ions which are prevalent in, and non-toxic to, most animals.

The source of electrical power that is electrically connected to the anode and the cathode can be of any variety. For instance, if the counter and donor electrodes are of dissimilar metals or have different half cell reactions, it is possible for the system to generate its own electrical power. Typical materials that provide a galvanic couple include a zinc-silver donor electrode and a silver chloride counter electrode. The zinc-silver combination will produce a potential of about one volt. When a galvanic couple is used, the donor electrode and counter electrode are integral portions of the power generating process. Such a galvanic couple powered system, absent some controlling means, activates automatically when body tissue and/or fluids form a complete circuit with the system. There exist numerous other examples of galvanic couple systems potentially useful in the present invention.

In some instances it may be necessary to augment the power supplied by the galvanic electrode couple, which may be accomplished with the use of a separate electrical power source. Such a power source is typically a battery or plurality of batteries, connected in series or in parallel, and positioned between the cathodic electrode and the anodic electrode such that one electrode is connected to one pole of the power source and the other electrode is connected to the opposite pole. Commonly, one or more 3 volt button cell batteries are suitable to power electrotransport devices. A preferred battery is a 3 volt lithium button cell battery.

The power source can include electronic circuitry for controlling the operation of the electrotransport device. Thus, the power source can include circuitry designed to permit the patient to manually turn the system on and off, such as with an on demand medication regime, or to turn the system on and off at some desired periodicity, for example, to match the natural or circadian patterns of the body. In addition, the control means can limit the number of doses that can be administered to the patient. A relatively simple controller or microprocessor could control the current as a function of time or could generate complex current waveforms such as pulses or sinusoidal waves. The control circuitry can also include a biosensor and some type of feedback system that monitors biosignals, provides an assessment of therapy, and adjusts the drug delivery accordingly.

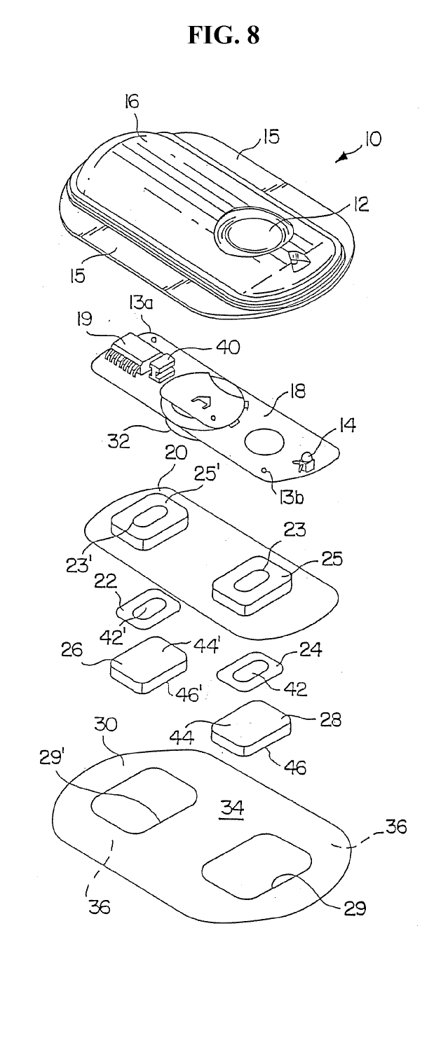

Reference is now made to FIG. 8, which depicts an exemplary electrotransport device that can be used in accordance with certain embodiment of the present invention. FIG. 8 shows a perspective exploded view of an electrotransport device 10 having an activation switch in the form of a push button switch 12 and a display in the form of a light emitting diode (LED) 14. Device 10 comprises an upper housing 16, a circuit board assembly 18, a lower housing 20, anode electrode 22, cathode electrode 24, anode reservoir 26, cathode reservoir 28 and skin-compatible adhesive 30. Upper housing 16 has lateral wings 15 that assist in holding device 10 on a patient's skin. Upper housing 16 is preferably composed of an injection moldable elastomer (e.g., ethylene vinyl acetate). Printed circuit board assembly 18 comprises an integrated circuit 19 coupled to discrete electrical components 40 and battery 32. Circuit board assembly 18 is attached to housing 16 by posts (not shown in FIG. 8) passing through openings 13a and 13b, the ends of the posts being heated/melted in order to heat stake the circuit board assembly 18 to the housing 16. Lower housing 20 is attached to the upper housing 16 by means of adhesive 30, the upper surface 34 of adhesive 30 being adhered to both lower housing 20 and upper housing 16 including the bottom surfaces of wings 15.

Shown (partially) on the underside of circuit board assembly 18 is a battery 32, which is preferably a button cell battery and most preferably a lithium cell. Other types of batteries may also be employed to power device 10.

The circuit outputs (not shown in FIG. 8) of the circuit board assembly 18 make electrical contact with the electrodes 24 and 22 through openings 23,23′ in the depressions 25,25′ formed in lower housing, by means of electrically conductive adhesive strips 42,42′. Electrodes 22 and 24, in turn, are in direct mechanical and electrical contact with the top sides 44′,44 of reservoirs 26 and 28. The bottom sides 46′,46 of reservoirs 26,28 contact the patient's skin through the openings 29′,29 in adhesive 30. Upon depression of push button switch 12, the electronic circuitry on circuit board assembly 18 delivers a predetermined DC current to the electrodes/reservoirs 22,26 and 24,28 for a delivery interval of predetermined length, e.g., about 10 minutes. Preferably, the device transmits to the user a visual and/or audible confirmation of the onset of the beneficial agent delivery, or bolus, interval by means of LED 14 becoming lit and/or an audible sound signal from, e.g., a “beeper”. The beneficial agent is then delivered through the patient's skin, e.g., on the arm, for the predetermined (e.g., 10 minute) delivery interval. In practice, a user receives feedback as to the onset of the beneficial agent delivery interval by visual (LED 14 becomes lit) and/or audible signals (a beep from the “beeper”).

Anodic electrode 22 is preferably comprised of silver and cathodic electrode 24 is preferably comprised of silver chloride. The donor reservoir is preferably comprised of polymer electrolyte. Electrodes 22, 24 and reservoirs 26, 28 are retained by lower housing 20.

The push button switch 12, the electronic circuitry on circuit board assembly 18 and the battery 32 are adhesively “sealed” between upper housing 16 and lower housing 20. Upper housing 16 is preferably composed of rubber or other elastomeric material. Lower housing 20 is preferably composed of a plastic or elastomeric sheet material (e.g., polyethylene) which can be easily molded to form depressions 25,25′ and cut to form openings 23,23′. The assembled device 10 is preferably water resistant (i.e., splash proof), and is most preferably waterproof. The system has a low profile that easily conforms to the body thereby allowing freedom of movement at, and around, the wearing site. The anode/drug reservoir 26 and the cathode/salt reservoir 28 are located on the skin-contacting side of device 10 and are sufficiently separated to prevent accidental electrical shorting during normal handling and use.

The device 10 adheres to the patient's body surface (e.g., skin) by means of a peripheral adhesive 30 which has upper side 34 and body-contacting side 36. The adhesive side 36 has adhesive properties which assures that the device 10 remains in place on the body during normal user activity, and yet permits reasonable removal after the predetermined (e.g., 24-hour) wear period. Upper adhesive side 34 adheres to lower housing 20 and retains the electrodes and drug reservoirs within housing depressions 25,25′ as well as retains lower housing 20 attached to upper housing 16.

The push button switch 12 is located on the top side of device 10 and is easily actuated through clothing. A double press of the push button switch 12 within a short period of time, e.g., three seconds, is preferably used to activate the device 10 for delivery of drug, thereby minimizing the likelihood of inadvertent actuation of the device 10.

Upon switch activation an audible alarm signals the start of beneficial agent delivery, at which time the circuit supplies a predetermined level of DC current to the electrodes/reservoirs for a predetermined (e.g., 10 minute) delivery interval. The LED 14 remains “on” throughout the delivery interval indicating that the device 10 is in an active beneficial agent delivery mode. The battery preferably has sufficient capacity to continuously power the device 10 at the predetermined level of DC current for the entire (e.g., 24 hour) wearing period.

The following examples are illustrative of certain embodiments of the invention and should not be considered to limit the scope of the invention.

### EXAMPLE 1

### In Vitro Skin Flux Experiments

Custom-built Delron horizontal diffusion cells were used for all in vitro skin flux experiments. A consumable Ag electrode with the same polarity as the drug was adhered to one end of the cell that functioned as the donor cell. The counter electrode was adhered at the opposite end. The electrodes were connected to a current generator (Maccor) that applied a direct current across the cell. The Maccor unit was a device with a built-in compliance voltage of up to 20 volts that maintained constant iontophoretic current.

For all in vitro electrotransport experiments, heat-separated human epidermis was used. In a typical experiment, the epidermis was punched out into suitable circles (2.38 cm) and refrigerated just prior to use. The skin was placed on a screen (2.38 cm) that fit into the midsection of the Delron housing assembly. Underneath the screen was a small reservoir that was 1.27 cm in diameter, 1.59 mm deep and could hold approximately 250 μl of receptor solution. The epidermis side of the skin was placed facing the drug-containing reservoir and the stratum corneum side faced the receptor reservoir. The receptor solution (saline, phosphate or other buffered solutions compatible with the drug) was continuously pumped through the reservoir via polymer tubing (Upchurch Scientific) connected to the end of a syringe/pump assembly. The pump could be set to any desired flow rate. In a typical experiment, a 1/10 diluted Dubeccos phosphate buffered saline receptor solution was used as the receiver fluid and was pumped into the receptor solution reservoir at 1 ml/hr. The drug containing reservoir was placed between the donor electrode and heat separated epidermis. A custom-built Delron spacer was used to encase the drug layer such that when the entire assembly was assembled together, the drug reservoir did not puncture the skin. Double-sided sticky tape was used to create a seal between all the Delron parts and to ensure that there were no leaks during the experiment. The entire assembly was placed between two heating blocks that were set at 32° C. to replicate skin temperature.

As the current was turned on at the onset of an experiment, the collection system (Hanson Research Microette™ collection system), which was interfaced with the experimental setup, was activated and served to collect the drug-containing receptor solution directly into HPLC vials. The collection system was programmed to collect samples at specified time intervals, depending upon the length of the experiment. In a typical experiment, the Hansen Microette™ collection system was programmed to collect samples every 1½ hour for 16 intervals over a 24 hour delivery experiment. The Hanson system was designed such that it could collect samples from up to twelve cells. From the cells, a piece of tubing transferred the receptor solution to the Microette™ and dispensed it into the HPLC vials, which were loaded onto a rotating wheel that could hold up to 144 vials, or 12 vials for each cell. The samples were then analyzed via HPLC to determine the efficiency of delivery of the drug in the formulation.

### EXAMPLE 2

### In Vitro Flux of Lidocaine HCl

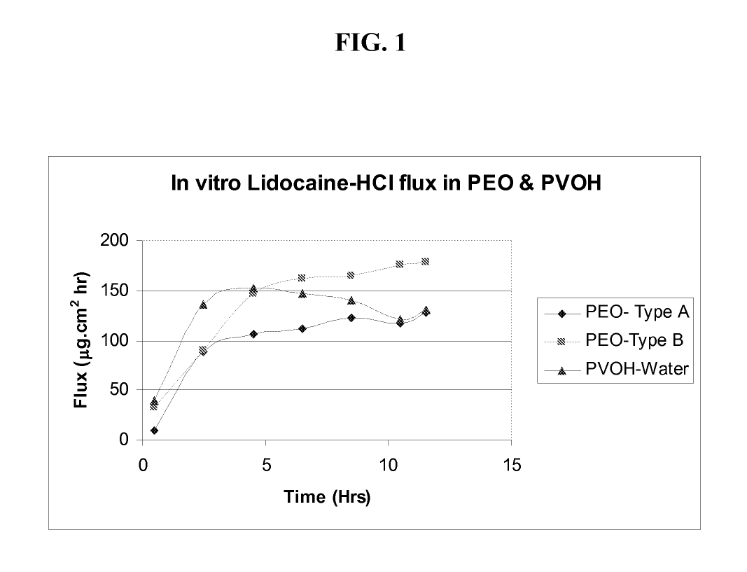

The in vitro flux of lidocaine HCl using various reservoirs was determined using the procedures described in Example 1, and the results are presented in FIG. 1. Experiments were performed using polyethylene oxide (PEO) reservoirs and polyvinylalcohol (PVOH) hydrogel reservoirs. The PEO reservoirs were either PEO films made using acetonitrile-ethanol as the solvent mixture (type A), or PEO films made using water (type B). When water was used as the solvent for casting the PEO films, residual water was removed by vacuum drying at 40° C. for 10 to 12 hours.

### EXAMPLE 3

### Determination of the Stability of Hydrocortisone Hemisuccinate in PVOH and PEO Matrices

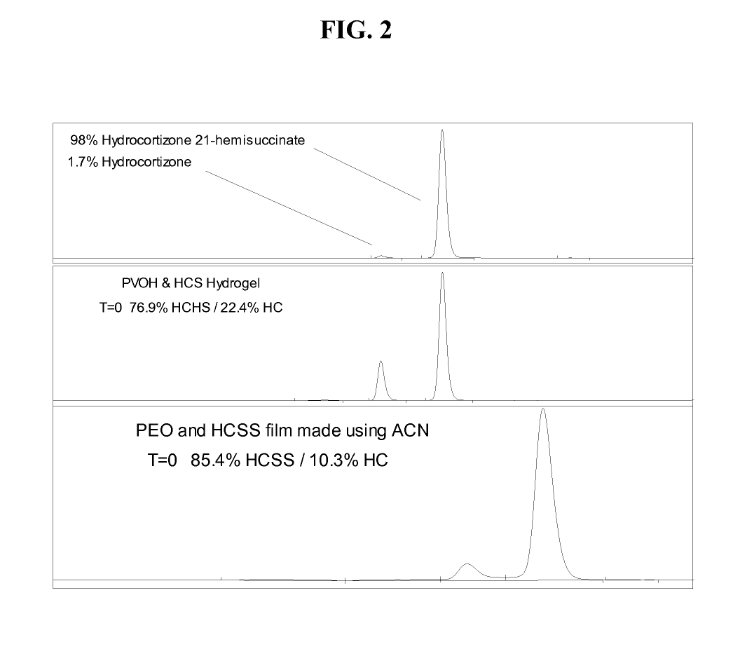

The stability of hydrocortisone hemisuccinate (HCHS), a hydrolytically unstable compound that forms hydrocortisone and succinic acid via hydrolysis, in PVOH hydrogels and PEO matrices was investigated. FIG. 2 shows an HPLC chromatogram of HCHS from a PVOH hydrogel and from a PEO film made using acetonitrile as the solvent. As seen from the figure, HCHS was more stable in the PEO matrix than in the PVOH hydrogel.

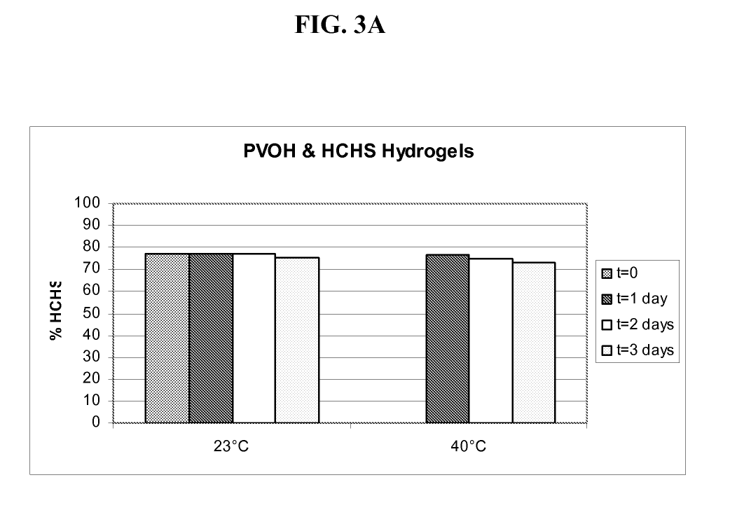

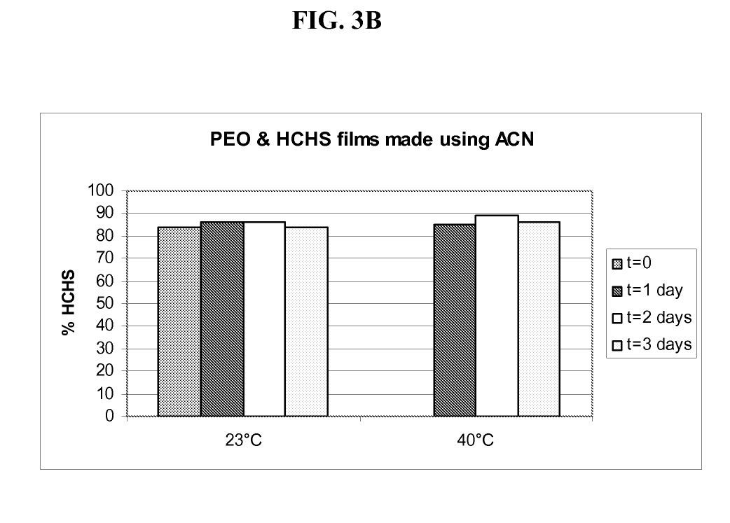

In a separate set of experiments (FIGS. 3A and 3B), the stability of HCHS in PVOH hydrogels and PEO matrices was examined at 23° C. and 40° C. over a period of three days. Improved stability of HCHS in PEO matrices was observed at 40° C.

### EXAMPLE 4

### Stability and In Vitro Flux of Apomorphine

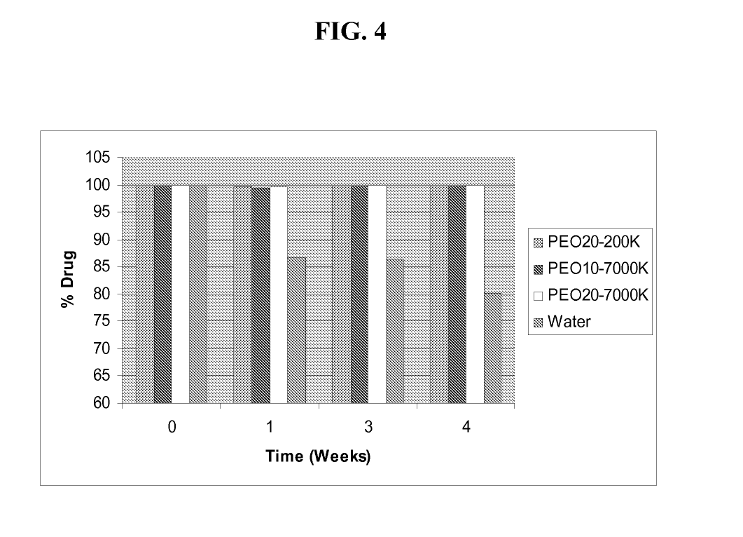

Apomorphine is highly unstable in aqueous solutions due to the presence of a cetechol moiety. Aqueous solutions of apomorphine undergo rapid oxidation in less than 30 minutes. Experiments were conducted to assess the stability of apomorphine in PEO matrices. The PEO films were cast using a 2:1 acetonitrile:methanol solvent mixture. As shown in FIG. 4, formulations of apomorphine containing PEO were stable for up to 4 weeks. The PEO used was either low formate or non-radiation crosslinked to prevent the formation of oxidative impurities.

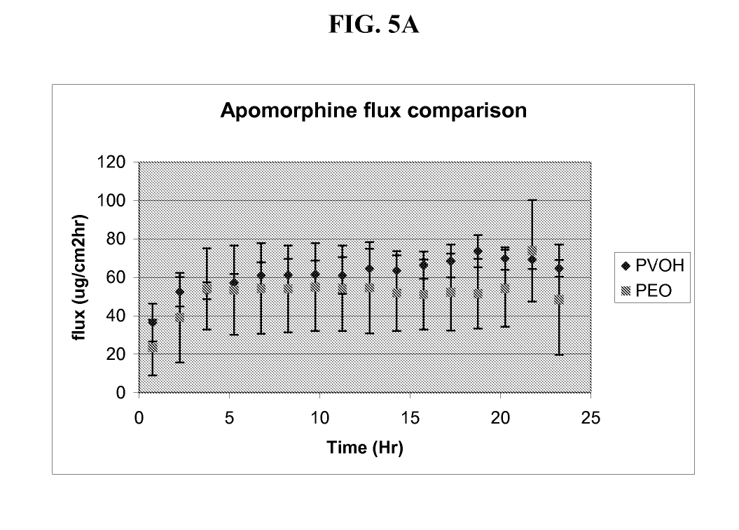

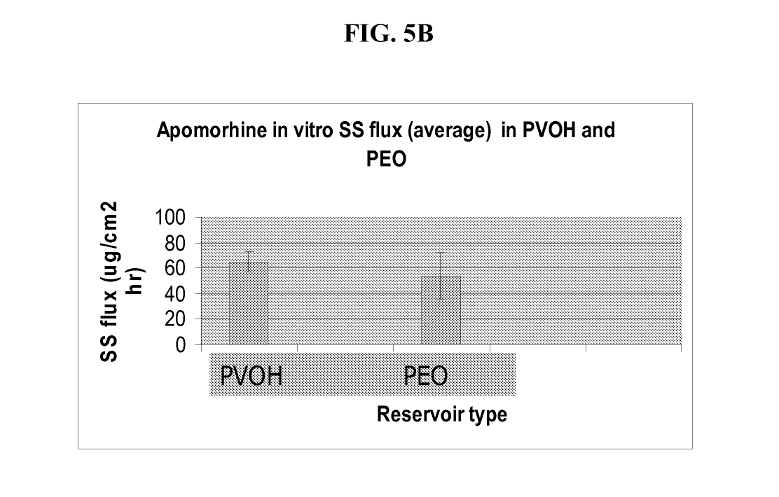

The in vitro flux of apomorphine was determined according to the procedures described in Example 1 using matrices composed of either PEO films or PVOH hydrogels. As seen in FIGS. 5A and 5B, the in vitro flux of apomorphine in both types of matrices was comparable.

### EXAMPLE 5

### In Vitro Flux of Fentanyl Hydrochloride

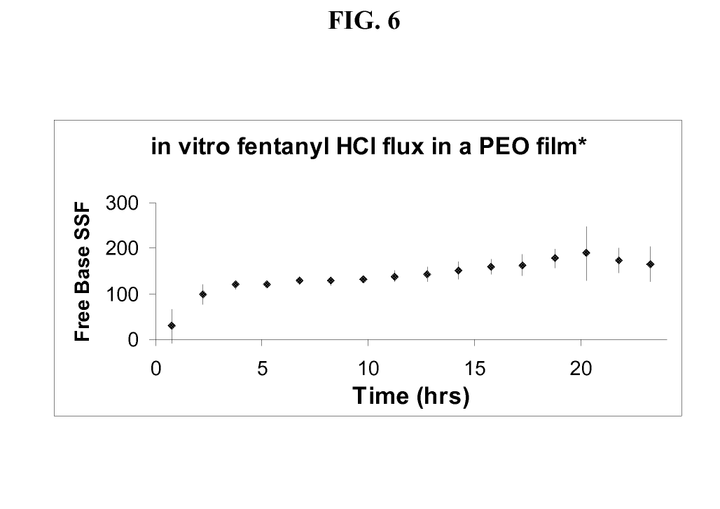

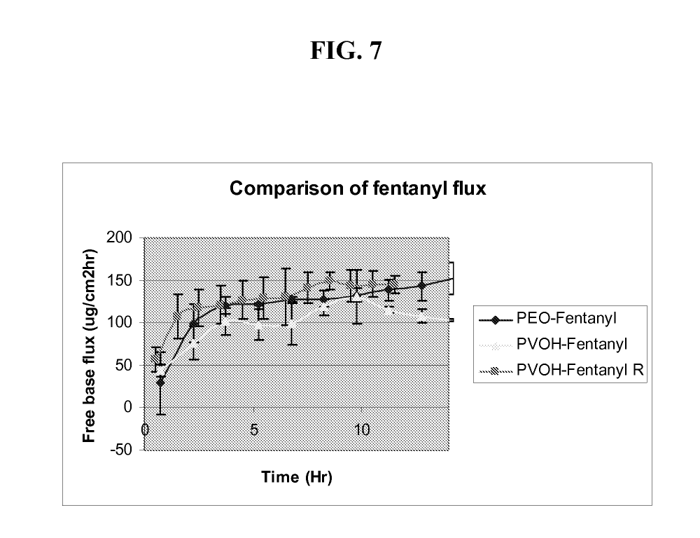

The in vitro flux of fentanyl HCl was determined using the procedures described in Example 1 using PEO film matrices, and the results are presented in FIG. 6. The in vitro flux of fentanyl HCl was also determined using the procedures described in Example 1 using PVOH matrices, and FIG. 7 shows a comparison of the in vitro flux of fentanyl HCl in a PEO matrix and in a PVOH hydrogel. The flux profile for the PEO matrices shows a quick onset to steady state followed by a transdermal steady state flux of approximately 120 μg/cm²hr, which is comparable to that obtained with the PVOH hydrogels.

The entire disclosure of each patent, patent application, and publication cited or described in this document is hereby incorporated herein by reference.

We claim:

1. A device for the electrotransport delivery of a beneficial agent comprising
a donor electrode assembly comprising a donor reservoir that comprises a polymer electrolyte that is substantially free of oxidants and impurities and contains a beneficial agent that remains stable during long-term storage of the device and during electrotransport;
a counter electrode assembly; and
a source of electrical power connected to the donor and counter electrode assemblies.

2. The device of claim 1 wherein the beneficial agent has a net positive charge.

3. The device of claim 1 wherein the beneficial agent is hydrolytically unstable.

4. The device of claim 3 wherein the beneficial agent is a hydrolytically unstable protein or polypeptide.

5. The device of claim 1 wherein the beneficial agent is lidocaine hydrochloride, hydrocortisone hemisuccinate, apomorphine hydrochloride, or fentanyl hydrochloride.

6. The device of claim 1 wherein the polymer electrolyte is in the form of a thin film.

7. The device of claim 6 wherein the polymer electrolyte is polyethylene oxide, a polysiloxane having a hydrophilic side chain, or a polyphosphazene having a hydrophilic side chain.

8. The device of claim 7 wherein the polymer electrolyte is polyethylene oxide.

9. A method for enhancing the stability of a hydrolytically unstable beneficial agent during long-term storage of a device for the electrotransport delivery of the hydrolytically unstable beneficial agent and during electrotransport delivery of the hydrolytically unstable beneficial agent comprising
providing a device for the electrotransport delivery of a hydrolytically unstable beneficial agent comprising
a donor electrode assembly comprising a donor reservoir that comprises a polymer electrolyte matrix that is substantially free of oxidants and impurities and contains the hydrolytically unstable beneficial agent;
a counter electrode assembly; and
a source of electrical power connected to the donor and counter electrode assemblies;
storing the device for up to six months; and
administering the hydrolytically unstable beneficial agent to a patient using the device,
wherein the hydrolytically unstable beneficial agent remains stable during storage and during electrotransport.

10. The method of claim 12 wherein the beneficial agent has a net positive charge.

11. The method of claim 12 wherein the beneficial agent is hydrolytically unstable.

12. The method of claim 14 wherein the beneficial agent is a hydrolytically unstable protein or polypeptide.

13. The method of claim 12 wherein the beneficial agent is lidocaine hydrochloride, hydrocortisone hemisuccinate, apomorphine hydrochloride, or fentanyl hydrochloride.

14. The method of claim 12 wherein the polymer electrolyte is in the form of a thin film.

15. The method of claim 17 wherein the polymer electrolyte is polyethylene oxide, a polysiloxane having a hydrophilic side chain, or a polyphosphazene having a hydrophilic side chain.

16. The method of claim 18 wherein the polymer electrolyte is polyethylene oxide.

17. The method of claim 12 wherein the device for delivery of a hydrolytically unstable beneficial agent is stored for one to three months prior to the electrotransport delivery of the hydrolytically unstable beneficial agent.
